(19) United States
(12) Patent Application Publication
(10) Pub. No.: US 2006/0064028 A1
(43) Pub. Date: Mar. 23, 2006

(54) Method for drawing up data which can be used to assess cognitive or sensomotor capabilities or capacities of people subjected to a test

(76) Inventor: Ognjen Amidzic, Glattbrugg (CH)

Correspondence Address: RANKIN, HILL, PORTER & CLARK LLP, 4080 ERIE STREET, WILLOUGHBY, OH, 44094-7836, US

(21) Appl. No.: 10/519,299

(22) PCT Filed: Jul. 1, 2002
(86) PCT No.: PCT/CH02/00354

§ 371 Date: Feb. 11, 2005

(51) Int. Cl. A61B 5/04; A61B 5/05

(52) U.S. Cl. 600/544; 600/410

(57) ABSTRACT

In order to draw up data which can be used to assess the cognitive or sensomotor capabilities or capacities of people subjected to a test, measuring samples collected by measuring methods known per se (e.g. magnetoencephalography or electro-encephalography) and representing the cerebral activities of the test person, are recorded in a synchronized manner with a sequence of different test situations which the test person faces. Relevant changes in activity are traced and localized from the recorded measuring samples. Groups are then formed based upon the locality of the relevant activity changes, each of the groups containing activity changes of a pre-determined cerebral region. The groups are interrelated and data describing the relation between the groups of relevant activity changes is prepared for the assessment, for example visualized or acoustically presented with experimentally determined limiting values or comparison data.

### BACKGROUND OF THE INVENTION

1. Field of the Invention

The invention is situated in the field of diagnostics in the widest sense and concerns a method according to the first independent claim. The method serves to generate data to be used for assessing cognitive and sensomotor capabilities and capacities of test persons.

2. Description of the Related Art

At the present time efficiency, i.e. the highest possible exploitation of all resources including human mental resources, is of great importance particularly in professional life. Therefore, expectations have risen enormously with regard to planning and interim new and re-adjustment of one's personal career and the careers of others. For the planning of a promising career it is important to be able to assess the cognitive and sensomotor capability of the person in question. One would like to operate in those areas in which one has capability enough for the highest possible achievements, one would like to chose employees who contribute a significant ability to the intended field of activity, one would like to avoid carreer termination due to insufficient capability.

This wish is complied with, according to the state-of-the-art technology, by a large selection of psychological tests a person is subjected to, the results of which tests are then interpreted by expert personnel. In most of these cases the tests comprise a test person to solve a problem by mental effort and then assessing the test person's solution to the problem. In many cases, however, it is not, or only insufficiently, possible to trace how the test person has arrived at the solution, and therefore what capabilities he or she has applied. This “how” is considerably less dependent on the nature of the test than the achieved result and therefore, would be particularly interesting and revealing for assessing the capabilities of the test person.

Various methods allowing mental activities to be observed are known from brain research. By such methods it is possible not just to chronologically track but also to locate, cerebral activities (e.g. neural discharge) that accompany e.g. sensory as well as cognitive activities. To this end the cerebral field of electric potential is measured, e.g. by magnetic encephalography or electro-encephalography. Such measurements allow to trace local potenial origins (potential sources). Magnetic encephalography registers electric potentials being induced by the cerebral electric field in a great number of inductors arranged all around the head of a test person. On the basis of the electric potentials registered at the same time in various inductors it is possible to determine the locations from where the field potentials come (to determine the cerebral location of potential sources), making it possible to allocate different cerebral functions to different cerebral regions.

Similar observations of cerebral activities as those based on magnetic encephalography or electro-encephalography are also possible e.g. by way of positron emission tomography (PET), with which essentially local and temporal changes of the cerebral blood circulation is observed, or by way of functional magnet resonance imaging (fMRI).

### SUMMARY OF THE INVENTION

The invention provides a method for generating data to be used for assessing cognitive and sensomotor capabilities or capacities of test persons. The generated data can be used e.g. to grade test persons in appropriate skill categories, to directly compare test persons with regard to a specific capability, or to assess a specific achievement of a test person (e.g. to assess whether a statement is a lie or not; lie detector).

### DETAILED DESCRIPTION OF THE PREFERRED EMBODIMENT

The method according to the invention builds a quantity of measuring data being collected from a test person by a per se known measuring method (e.g. magnetic encephalography, electro-encephalography, PET, FMRI). Such quantity of measuring data which consists of a large number of meansuring samples constitutes an image of the cerebral activities of the test person. When using magnetic encephalography each measuring sample consists of the inducted volatages recorded from the inductors in a given moment in time (sample point). Time frames in which measuring data are registered are synchronised with a succession of different test situations which are presented to the test person. Advantageously each such time frame begins essentially simultaneously with a new test situation and is of a predetermined length compatible with the nature of the test situation. The test situations contain visual, acoustic, or somatic sensory stimuli addressing the experiences of the test person in the area of the capabilities or capacities to be assessed.

From the registered measuring samples relevant changes in cerebral activity are traced and located. In other words, a cerebral region in which there is a change in cerebral activity is recorded, if the activity change is relevant enough for assuming that the presented test situation is the reason for the activity change. From measuring samples collected by magnetic encephalography or electro-encephalography, the potential sources (locations of the origin of the potential) are calculated in order to trace and locate the relevant activity changes. The measuring samples and/or the data generated from the measuring data and describing the potential sources are filtered in order to trace relevant sources only.

The relevant changes in cerebral activity located from the measuring samples are then assigned to a plurality of groups, each group containig the activity changes allocated to predetermined cerebral region. This is done either generally, i.e. for all traced relevant activity changes, or for each individual time frame, or for each individual test situation, or for groups of test situations related by the same subject matter. Therein each group of relevant activity changes contains relevant changes, which occurred in the one predetermined cerebral region. The groups of relevant activity changes allocated to the specified cerebral regions are then interrelated.

It is found that such group relations, generated from the originally collected quantity of measuring data registered for a sucession of different test situations of a given nature, can be used directly for the comparison of different test persons with regard to cognitive or sensomotor capabilities. Therein the capabilities to be assessed determine in particular the nature of the test situations, the synchronisation and the duration of the time frames for registering the measuring samples, as well as the definition of the different cerebral regions. It is further found that with the aid of such group relations, if established for each time frame, or for each test situation, or for groups of test situations related to a similar matter, very specific cognitive or sensomotor capacities of test persons can be assessed. It is possible e.g. to compare such group relations with the test person's comments on the test situation and thereby to deduce a content of truth in the sense of a lie detector.

In order to assess capabilities of test persons, the data representing the group relations are e.g. compared with calibration curves or threshold values established by experiment, thus the test persons can be graded regarding predetermined categories of capability, or the group relation data of various test persons can be directly compared with each other.

The method according to the invention comprises essentially four steps:

(1) Registering measuring samples which image cerebral activity of the test person in time frames which are synchronized with a succession of different test situations being presented to the test person;

(2) Tracing and locating relevant changes in the cerebral activities from the registered measuring samples (for all time frames or for each time frame or for groups of time frames);

(3) Generating a number of groups of relevant changes of cerebral activity, wherein each group is allocated to a predetermined cerebral region, i.e. contains traced relevant activity changes located in this cerebral region;

(4) Interrelating in a predetermined manner the groups allocated to the different cerebral regions and preparing data which define the group relation for the assessment.

The invention is based on the discovery of a correlation between the Elotable of chess players and a relation between the number of potential sources in the frontal, occipital and parietal lobes (memory) and the number of potenial sources in the temporal lobe, hippocampus and limbic system (encoding) traced during games of chess. The discovery was made using magnetic encephalography for data collection [Amidzic, O. et al., Nature, vol. 412, 9. Aug. 2001].

An examplary embodiment of the method according to the invention, to be described in more detail below, serves the assessment of test persons with regard to their capability of using experience in a specified area for solving problems in this area (assessment of expertise). Extensive use of experience indicates a high capability in greatly varied fields of activity, in particular in the strategic sector.

A succession of different test situations is presented to a test person. The test situations represent different problems, e.g. presented to the test person in visualized form, which can be solved with the expertise to be assessed. Measuring samples imaging the cerebral activity of the test person are registered during time frames which begin with each presentation, or immediately thereafter, and last from 0.1 to 3000 seconds. The measuring samples are gained by e.g. measuring cerebral field potentials in a per se known manner (magnetic encephalography) and with a sampling frequency of 10 to 5000 hertz (preferably 20 to 1400 hertz).

From the registered measuring samples, relevant changes in cerebral activity are reaced and located by subjecting observed activity changes to an admittance test suitable for the applied model (filtering), by isolating the relevant ones of the changes, and by determining the place of occurrence in the brain of such relevant activity changes. This means that from the cerebral field potentials measured by magnetic encephalography, potential sources are calculated. Sources in a frequency range of 4 to 80 hertz (preferably 20 to 40 hertz, cognitive cerebral activity) and with a “goodness of fit” larger than 90% are isolated and used further for the assessment. If applicable, the intensities of the sources can also be subjected to a filtering process.

The relevant activity changes traced and located by the admittance test (e.g. potential sources calculated from data registered by magnetic encephalography) are then allocated to different groups based on the cerebral region (locality) of their occurence. For an assessment regarding expertise, a first cerebral region comprises the frontal, occipital and parietal lobes (memory) and a second cerebral region comprises the temporal lobe, the hippocampus and the limbic system (encoding).

The groups of relevant changes in cerebral activity allocated to different cerebral regions are interrelated. E.g. the relative numbers of relevant changes observed in the specified cerebral regions are determined, in the two cerebral regions named above, which are attributed to memory and encoding. A large proportion of relevant cerebral activity changes in the cerebral region of the frontal, occipital and parietal lobes indicate a high level of expertise (extensive use of the appropriate experience). A large proportion of relevant activity changes in the cerebral region of the temporal lobe, hippocampus and limbic system indicate a low level of expertise.

The data describing the aforementioned group relation (relation data) are prepared for the assessment, by e.g. being visualized together with comparable data, calibration curves and/or grades, or they are directly processed into a verbal assessment.

The embodiment of the method according to the invention described above can in a slightly modified way also be used for testing whether a test person connects certain test situations with personal experiences or not, e.g. whether they have or have not already seen images presented to them. Thus the method can be used e.g. in the function of a lie detector if the relation data are connected to statements made by the test person. Therein the process is essentially the same as described above for the assessment of a capability, except that the analysis of the measuring samples is not carried out generally, i.e. using all available time frames or test situations respectively, but is carried out per individual time frame, or per individual test situation, or per group of test situations related by a predetermined subject matter. Thus the test person is confronted e.g. with a series of portraits or situation images. In each time frame following a presentation, measuring samples are registered and relevant cerebral activity changes are then detected and located. A large proportion of such relevant changes to be assigned to the cerebral region of frontal, occipital and parietal lobes, or a small proportion of relevant activity changes to be assigned to the cerebral region temporal lobe, hippocampus and limbic system, indicate that the test situation addresses experiences, i.e. that the person is familiar with the depicted persons or situations.

Further embodiments of the method according to the invention differ from the above described embodiment e.g. regarding the synchronization of sampling and test situation presentation, regarding admittance tests for determining the relevant ones of the activity changes, regarding the cerebral regions or their delimitation, and/or regarding calculation of the relation data. E.g. the time frames for sampling are distanced from the moment in which the test preson is first presented with the test situation and/or it is extended, in order to rather assess the way of solving the presented problem than assessing the first cerebral access to material useful for such solution. It is possible to distinguish between more than two, but smaller cerebral regions, and it is possible to use e.g. the summed-up intensities of the traced activity changes, or similar quantities, instead of their number for calculating the required group relation data.

The optimal parameters of the method according to the invention, including the selection of test situations and their chronological succession, are to be established by experiment for each application. The same is valid for determining grades and/or threshold values for grading or comparing test persons.

The device for carrying out the method according to the invention is in essence a data processing system. This data processing system comprises an interface for input of a large number of measuring samples collected by a system for measuring the cerebral activities of a test person. The installation further comprises the following means: a means for presenting to the test person a succession of different test situations; a means for synchronizing the named presentation with the registration of measuring samples; a means for tracing and locating relevant activity changes from the registered measuring samples; a means for assigning the relevant activity changes to a plurality of groups by allocating the location of their occurende to predetermined cerebral regions; a means for generating data defining a relation between the groups of relevant activity changes; and a means for preparing the relation data for the assessment.

The aforementioned data processing system comprises e.g. a suitably programmed computer unit and a display screen or loudspeaker for the visual or acoustic presentation of the test situations to the test person. Also to be protected is a storage medium with a program stored therein which program when installed in a computer furnished with a known system software enables the computer to execute the method according to the invention.

1. A method for generating data to be used for assessing cognitive or sensomotor capabilities or capacities of a test person, comprising the steps of:
registering measured samples, which are collected by a per se known measuring means and image cerebral activities of the test person, during time frames which are synchronized with a presentation of a succession of test situations to the test person;
tracing and locating, from the collected measuring samples, relevant changes in cerebral activity in the brain of the test person;
assigning relevant activity changes to a plurality of groups, wherein each group contains the relevant activity changes located in a predetermined cerebral region;
interrelating groups of relevant activity changes; and,
preparing data defining the relation between the groups for assessment.

2. The method according to claim 1, wherein, the step of tracing and locating relevant activity changes, the step of assigning relevant activity changes to groups and the step of interrelating the groups are carried out for at least one of: a sum of the measuring samples registered in all time frames, each individual time frame, and a group of time frames each.

3. The method according to claim 1, wherein the time frames for registering of measuring samples are synchronized with a succession of test situations such that a time frame begins at a start of each test situation of the succession.

4. The method according to claim 3, wherein, each time frame has a length of between 0.1 and 3000 seconds.

5. The method according to claim 1, wherein the test situations are problems which are solvable using a specific experience and which are presented visually or acoustically.

6. The method according to claim 1, wherein the test situations are images or other situations aimed at a possible experience of the test person.

7. The method according to claim 1, wherein the relevant activity changes are traced by filtering and/or by an admittance test.

8. The method according to claim 1, wherein the cerebral regions of the step of assigning relevant activity changes to groups are the cerebral region of the frontal, occipital and parietal lobes and the cerebral region of the temporal lobe, the hippocampus, and the limbic system.

9. The method according to claim 1, wherein the step of interrelating the groups of relevant activity changes comprises the step of determining a proportion of numbers of relevant activity changes in the groups.

10. The method according to claim 1, wherein the step of preparing the data for assessment comprises presenting the data describing the group relation visually or acoustically together with experimentally determined assessement grades, comparative data and/or threshold values.

11. The method according to claim 1, wherein the measuring samples are collected by magnetic encephalography or electro-encephalography and wherein the relevant activity changes are potential sources of a frequency range of 4 to 80 hertz and with a goodness of fit of more than 90%.

12. The method according to claim 11, wherein the measuring samples are recorded with a frequency of 10 to 5000 hertz.

13. A data processing system for generating data to be used for assessing cognitive or sensomotor capabilities or capacities of a test person, comprising:
an interface for input of measuring samples collected by a per se known method, the measuring samples imaging cerebral activities of the test person,
means for presenting a succession of different test situations to the test person,
means for synchronizing the succession of test situations with time frames in which measuring samples are registered,
means for tracing and locating relevant activity changes from the registered measuring samples,
means for assigning the relevant activity changes to a plurality of groups based on the location of the activity changes in a plurality of different predetermined cerebral regions,
means for interrelating the groups of relevant activity changes, and
means for preparing data describing the relation between the groups for assessment.

14. The data processing system according to claim 13, wherein the means for tracing and locating relevant activity changes comprises means for filtering and/or a means for subjecting the traced activity changes to an admittance test.

15. The data processing system according to claim 13, further comprising a display screen or a loudspeaker for visual or acoustic presentation of the succession of test situations and/or for the step of preparing the data reflecting the group relation for the assessment.

16. A storage medium with a program code stored therein, said storage medium, when inserted into a computer, induces the computer to perform the method of claim 1.

17. Use of the method according to claim 1 for assessing test persons with regard to their ability to make use of their experience.

18. Use of the method according to claim 1 as a lie detector.
